(19) United States
(12) Patent Application Publication
(10) Pub. No.: US 2004/0116998 A1
(43) Pub. Date: Jun. 17, 2004

(54) Endovascular prosthesis

(76) Inventors: Raimund Erbel, Essen (DE); Donald r Ricci, Vancouver (CA); Ian M Penn, Vancouver (CA); George A Shukov, Los Altos, CA (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/416,819

(22) PCT Filed: Nov. 19, 2001
(86) PCT No.: PCT/CA01/01587

(51) Int. Cl. A61F 2/06

(52) U.S. Cl. 623/1.13; 623/1.15

(57) ABSTRACT

An endovascular prosthesis for implantation in a body passageway. The prosthesis comprises an elongate tubular wall comprising an annular portion for occlusion of a section of the body passageway. The annular portion comprises a first porous section and a non-porous section. In one embodiment, the non-porous section may comprise a cover material. In another embodiment, the non-porous section may comprise a series of slits, microcuts, slots, apertures and the like which serve to impede the flow of bodily fluid therethrough resulting in occlusion of an aortic disease condition located exteriorly adjacent to the deployed prosthesis.

### TECHNICAL FIELD

In one of its aspects, the present invention relates to an endovascular prosthesis. In another of its aspects, the present invention relates to a method of treating an aortic disease condition in a patient.

### BACKGROUND ART

Stents are generally known. Indeed, the term “stent” has been used interchangeably with terms such as “intraluminal vascular graft” and “expandable prosthesis”. As used throughout this specification the term “stent” is intended to have a broad meaning and encompasses any expandable prosthetic device for implantation in a body passageway (e.g., a lumen or artery).

In the past ten years, the use of stents has attracted an increasing amount of attention due the potential of these devices to be used, in certain cases, as an alternative to surgery. Generally, a stent is used to obtain and maintain the patency of the body passageway while maintaining the integrity of the passageway. As used in this specification, the term “body passageway” is intended to have a broad meaning and encompasses any duct (e.g., natural or iatrogenic) within the human body and can include a member selected from the group comprising: blood vessels, respiratory ducts, gastrointestinal ducts and the like.

Stent development has evolved to the point where the vast majority of currently available stents rely on controlled plastic deformation of the entire structure of the stent at the target body passageway so that only sufficient force to maintain the patency of the body passageway is applied during expansion of the stent.

Generally, in many of these systems, a stent, in association with a balloon, is delivered to the target area of the body passageway by a catheter system. Once the stent has been properly located (for example, for intravascular implantation the target area of the vessel can be filled with a contrast medium to facilitate visualization during fluoroscopy), the balloon is expanded thereby plastically deforming the entire structure of the stent so that the latter is urged in place against the body passageway. As indicated above, the amount of force applied is at least that necessary to expand the stent (i.e., the applied the force exceeds the minimum force above which the stent material will undergo plastic deformation) while maintaining the patency of the body passageway. At this point, the balloon is deflated and withdrawn within the catheter, and is subsequently removed. Ideally, the stent will remain in place and maintain the target area of the body passageway substantially free of blockage (or narrowing).

An alternate approach is the so-called “self-expanding” stents. In this approach, the stent is compressed in a sheath. The stent/sheath combination is delivered to the body passageway of interest and, thereafter, the sheath is retracted. As the stent is exposed, potential energy stored in the stent is converted to kinetic energy and the stent expands. This is a common approach with conventional wire stents and nitinol stents.

See, for example, any of the following patents:

U.S. Pat. No. 4,733,665 (Palmaz),

U.S. Pat. No. 4,739,762 (Palmaz),

U.S. Pat. No. 4,800,882 (Gianturco),

U.S. Pat. No. 4,907,336 (Gianturco),

U.S. Pat. No. 5,035,706 (Gianturco et al.),

U.S. Pat. No. 5,037,392 (Hillstead),

U.S. Pat. No. 5,041,126 (Gianturco),

U.S. Pat. No. 5,102,417 (Palmaz),

U.S. Pat. No. 5,147,385 (Beck et al.),

U.S. Pat. No. 5,282,824 (Gianturco),

U.S. Pat. No. 5,316,023 (Palmaz et al.),

U.S. Pat. No. 5,755,771 (Penn et al.),

U.S. Pat. No. 5,906,640 (Penn et al.),

U.S. Pat. No. 6,217,608 (Penn et al.),

U.S. Pat. No. 6,183,506 (Penn et al.),

Canadian patent 1,239,755 (Wallsten), and

Canadian patent 1,245,527 (Gianturco et al.), for a discussion on some previous stent designs and deployment systems.

To date, most stent development has focused on the so-called coronary stents. While a number of advances in art of coronary stent development have been made, there is room for improvement.

One area which has received little or no attention is the area of endovascular treatment of aortic disease. At this point it is useful to review diseases of the aorta.

Aortic diseases contribute to the high overall cardiovascular mortality. Relatively new imaging modalities (e.g., transesophageal echocardiography, magnetic resonance tomography, helical computed tomography, electron beam computed tomography) have been introduced during the last decade. These new imaging techniques facilitate better and/or earlier diagnosis of aortic diseases, even in emergency situations. These new imaging techniques have had an effect on patient management during recent years allowing more rapid diagnosis and decision making.

Generally, aortic disease is caused by mechanisms which weaken the strength of the aortic wall, particularly, the aortic media. Such wall weakening leads to higher wall stress, which can induce aortic dilatation and aneurysm formation, eventually resulting in aortic dissection or rupture. The various categories of aortic disease are summarized in FIG. 1.

Diseases of the aorta are a significant problem in medicine. There are two general approaches: drug treatment and surgery. Drug treatment is used to lower blood pressure—this approach is disadvantageous since, at best, it modulates the effect of the disease while still leaving the patient at significant risk. Surgery is disadvantageous due to the high mortality and morbidity even in centers of excellence. The increasing age of the population is resulting in an increased incidence of aortic disease as it is a degenerative disease. Further, aortic stiffness increases with age thereby reducing coronary and other artery perfusion.

There are three (3) indications of aortic disease which are regularly of clinical interest: (1) aortic dissection, (2) blunt chest trauma (with consequential trauma to the aorta), and (3) aortic sclerosis.

Aortic dissection is known to occur in approximately 15-20 cases/1 million inhabitants/year with a mortality of 50% in the first year and 5% per hour for the first 5 hours after the onset of symptoms. It results in a splitting of the aortic wall, a bleeding into the wall with formation of a true and false (new) lumen separated by a flap called “intima” with tear or “rupture point”. In patients with involvement of the ascending aorta, surgery is performed and drug treatment preferred in patients with involvement of the descending aorta. As stated above, despite surgery, mortality is still high. The main problem is the organ perfusion of the abdomen which results in shock and multiorgan failure.

Relatively recent studies have demonstrated that intramural hemorrhage, intramural hematoma and aortic ulcer may be signs of evolving dissections or dissection subtypes. Currently, the various forms of dissection may be classified as follows:

Class 1 (FIG. 2a): Classical aortic dissection with an intimal flap between true and false lumen;

Class 2 (FIG. 2b): Medial disruption with formation of intramural hematoma/hemorrhage;

Class 3 (FIG. 2c): Discrete/subtle dissection without hematoma, eccentric bulge at tear site;

Class 4 (FIG. 2d): Plaque rupture leading to aortic ulceration, penetrating aortic atherosclerotic ulcer with surrounding hematoma, usually subadventitial; and

Class 5 (FIG. 2e): Iatrogenic and traumatic dissection.

Each of these classes of dissection can be seen in their acute and chronic stages; chronic dissections are considered to be present if more than 14 days have elapsed since the acute event.

Classic Aortic Dissection (Class 1—FIG. 2a)

Acute aortic dissection is characterized by the rapid development of an intimal flap separating a true lumen and false lumen. Due to the pressure difference the true lumen is usually smaller than the false lumen. Intimal flap tears characterize communicating dissections. However, tears are not always found and non-communicating dissections are not uncommon. The dissection can spread from diseased segments of the aortic wall in an antegrate or retrograde fashion, involving side branches and causing other complications.

Intramural Hematoma/Hemorrhage (Class 2—FIG. 2b)

An intramural hematoma is believed to be the initial lesion in the majority of cases of cystic medial degeneration leading to aortic dissection in which the intimal tear seems to be secondary to preceding intramural dissection. Intramural hematoma may be the result of ruptured normal-appearing vasa vasorum which are not supported by the surrounding aortic media or the result of rupture of diseased vasa vasorum. As a dissecting hematoma extends along the aorta the weakened inner wall is subjected to the elongating force of the diastolic recoil. Differences in elasticity between the aortic fibrous adventitia and the inner more elastic media may play an additional role.

In autopsy studies, dissecting aneurysms without tears have been found in up to 12% of 311 autopsies. Others studies have reported an incidence of 4% in 505 cases. In a series of sudden deaths, 67% of patients with dissections did not have tears. The incidence of intramural hemorrhage and hematoma in patients with suspected aortic dissection, as observed by various new imaging techniques, seems to be in the range of 10-30%.

There are two distinct types of intramural hematoma and hemorrhage. Type I intramural hematoma and hemorrhage shows a smooth inner aortic lumen, the diameter is usually less than 3.5 cm, and the wall thickness greater than 0.5 cm. Echo free spaces (seen echocardiographically) as a sign of intramural hematoma are found in only □of the patients. The mean longitudinal extent of the hematoma is about 11 cm and the echo free spaces show minimal or no signs of flow.

Type II intramural hematoma and hemorrhage occurs in aortic arteriosclerosis. A rough inner aortic surface with severe aortic sclerosis is characteristic, the aorta is dilated to more than 3.5 cm and calcium deposits are frequently found. Mean wall thickness is 1.3 cm with a range of from about 0.6 to about 4 cm, and echo free spaces are found in 70% of the patients studied. The longitudinal extension has a similar range as in Type I hematoma, usually about 11 cm. Intramural hemorrhages are more often found in the descending than in the ascending aorta.

The fact that intramural hemorrhage and hematoma can lead to aortic dissection has only be demonstrated in follow-up studies. Acute aortic dissection as a consequence of intramural hemorrhage and hematoma develops in from about 28% to about 47% of the patients. It is associated with aortic rupture in from about 21% to about 47%; and regression is seen in about 10% of the patients.

Subtle-Discrete Aortic Dissection (Class 3—FIG. 2c)

The structural weakness can either lead to clinically undetected disease or minor forms of aortic dissection. Subtle dissection has been described as a partial stellate or linear tear of the vessel wall, covered by thrombus. After the partial tear forms a scar, this constellation is called abortive, discrete dissection. Partial ruptures of the inner layer of the aorta allow the blood to enter the already damaged media and thus cause dissection of the aortic wall, eventually leading to a second lumen within the wall, to a rupture or healing during follow-up.

Plaque Rupture/Ulceration (Class 4—FIG. 2d)

Ulceration of atherosclerotic aortic plagues can lead to aortic dissection or aortic perforation. This was first observed by computed tomography. Innovations in imaging techniques (e.g., intravascular ultrasound, spiral computed tomography and magnetic resonance imaging) provide new insight. The ability to diagnose aortic ulceration has thereby been improved and further insight into the pathophysiology of this condition was gained. The ulcers seem to affect the descending thoracic aorta, as well as the abdominal aorta, and are usually not associated with an extensive longitudinal propagation or branch vessel compromise. Valvular, pericardial or other vascular complications seem to be rare. The ulcer may penetrate beyond the intimal border, often with an nipple-like projection with subjacent Type II intramural hematoma formation. The continuous erosion of the atherosclerotic plaque may eventually violate the internal elastic membrane. False aneurysms, aortic rupture or dissections may occur.

Aortic sclerosis is normally divided into four grades from thickening of the intima (Grade I) up to the development of free floating thrombi (Grade IV) with the danger of embolism. In elderly patients, the incidence of the Grade IV aortic sclerosis is increasing. This has lead to a significant occurrence of stroke in patients. Thus, if a treatment of aortic sclerosis Grade IV with thrombi free floating in the aortic lumen could be developed, this would likely obviate or mitigate the consequential occurrence of stroke.

Currently, there is no reliable treatment approach for aortic sclerosis, particularly the Grade IV type. Anticoagulation is a known approach, however this treatment must be accepted with the danger of hemorrhagic strokes, particularly in the older patients Further, the therapy is very difficult to monitor. Surgery is very complicated and has a high mortality and morbidity. Currently, surgery is not seen as a desirable altenative to anticoagulation therapy.

Traumatic/Iatrogenic Aortic Dissection (Class 5—Fissure 2e)

Blunt chest trauma usually causes dissection of the ascending aorta and/or the region of the ligamentum Botalli at the aortic isthmus. Iatrogenic dissection of the aorta may rarely occur during heart catheterization. It is regularly seen following angioplasty of an aortic coarctation, but can also be observed after cross clamping of the aorta and after the use of intraaortic balloon pumping. Most catheter-induced dissections are retrograde dissections. They will usually decrease in size as the false lumen thromboses. Proximal progression of the coronary dissection into the aortic root may be observed. In blunt chest trauma, the large acceleration of the aorta is leading to an intimal, medial or transsection of the aorta particularly at the adjunction at the aortic arch and the descending aorta (15-20% of blunt chest trauma cases are related to aortic injury). As a consequence of this blunt chest trauma, mediastinal hematoma can occur with abrupt death of the patient. The blunt chest trauma is known to occur in accidents involving heavy motorcycles and cars, as well as in other chest traumas. The diagnosis is very difficult but has been improved by transesophageal echocardiography. Typically, the damage to the aorta is limited to a small portion comprising 3 cm to 5 cm of the aorta. Conventionally, surgery was the only treatment to stabilize these patients. A mortality, rate of 90% has been seen if surgery was not timely preformed. Even if surgery was timely performed, there is a significant mortality rate.

Most prior art attempts to improve surgical techniques to treat aortic dissection have not be particularly successful.

It is also worth pointing out that the so-call “stent grafts” are not well suited for treating diseases of the aorta. As is known in the art, a stent graft is a prosthesis having a stent portion and a cover portion, each of which are tubular. In use, they will cover the entire interior surface of the lumen in which they are deployed. While this is not problematic in certain coronary applications, this can lead to catastrophic results in the treatment of aortic diseases since there is a significant likelihood of side branch arterial occlusion by the graft portion. A block of such arteries supplying the spinal cord can occur leading to paraplegia which has been observed when current stent grafts have been used in the treatment of aortic dissection.

Thus, despite the advances made in the art, there is still a need for an endovascular prosthesis capable obviates or mitigates at least one of the above-mentioned disadvantages of the prior art.

### DISCLOSURE OF THE INVENTION

It is an object of the present invention to provide a novel endovascular prosthesis which obviates or mitigates at least one of the above-mentioned disadvantages of the prior art.

Accordingly, in one of its aspects, the present invention provides an endovascular prosthesis for implantation in a boded passageway, the prosthesis comprising a tubular wall, the tubular wall comprising an annular portion for occlusion of a section of the body passageway, the annular portion comprising a first porous section and a non-porous section.

In another of its aspects, the present invention provides a method for endovascular blocking of an aortic disease condition in a body passageway of a patient with an endovascular prosthesis comprising an elongate tubular wall, the tubular wall comprising an annular portion for occlusion of the aortic disease condition, the annular portion comprising a first porous section and a non-porous section, the method comprising the steps of:

disposing the prosthesis in a catheter;

inserting the prosthesis and catheter within a body passageway by catheterization of the body passageway;

translating the prosthesis and catheter to a target body passageway at which the aortic disease condition is located;

positioning non-porous section such that it is substantially aligned with the aortic disease condition;

exerting a radially outward expansive force on the tubular wall such that the tubular wall is urged against the target body passageway; and

urging the non-porous section against the aortic disease condition thereby blocking the aortic disease condition.

Thus, the preferred form of the present endovascular prosthesis device is a stent system with partially radially, covered by a non-porous or graft material.

Generally, the present prosthesis can be advantageously used to treat the indications of aortic disease referred to hereinabove.

With reference to aortic dissection, the present prosthesis normally will be implanted at the side of the intima tear in order to block the flow from the true lumen into the false lumen at the dissection connection. The present prosthesis may be advantageously used in dissection of the descending part of the aorta.

A feature of the present endovascular prosthesis is that it has only a partial, radial non-porous or graft covering. Placement and positioning of the device can be facilitated by intravascular ultrasound and transesophageal echocardiography blocking the tear and while obviating or mitigating covering the entire aortic wall—e.g., the portion of the aortic wall possibly containing important side branches.

An advantage of the present endovascular prosthesis is that it allows flow from the proximal to the distal aorta even during the implantation of the device due to the unique design. In contrast, conventional stent grafts must be used with the concurrent danger of abrupt rise of blood pressure leading to an extension and enlargement of the dissection.

The present endovascular prosthesis may be used advantageously to block the tear, thereby obviating or mitigating flow from the true lumen to the false lumen. Thus, the healing process begins which, in the successful cases, will lead during follow-up within 6 months to total obliteration of the false lumen and strengthening of the aortic wall. In addition the pressure in the false lumen is reduced or eliminated and thereby, the true lumen can expand and improve the organ perfusion.

When properly deployed, the present endovascular prosthesis will protect the diseased part of the aorta, so that little or no blood is escapes from the lumen to the mediastinum and thereby, the patient is stabilised in the acute phase of the aortic injury. Using intravascular ultrasound and transesophageal echocardiography, the present endovascular prosthesis may be appropriately navigated to block the damage of the aorta. Again as in treatment of aortic dissection, it is important to avoid blockage of multiple arteries which are supplying the spinal cord since this can lead to paraplegia with enormous consequences for the patient.

Indeed, to the knowledge of the present inventors, the present endovascular device is the first such device to be useful in reliable treatment of aortic diseases. Thus, with the present endovascular device, blockage of the aortic flow is obviated or mitigated and abrupt blood pressure increases (which could lead to a fatal event) are avoided. Further, since the present device may be deployed endovascularly (i.e., non-surgically), it is generally safer for the patient and is less of a burden on public health systems.

The present endovascular prosthesis may be used advantageously to wrap the intimal flaps and thrombi to the aortic wall and thereby obviate or mitigate the danger of stroke and emboli without the need for anticoagulation. As the prosthesis covers only a radial portion of the aortic circumference, blocking of side arteries, which are supplying the spinal cord, is obviated or mitigated. As the present prosthesis is open and not blocking the flow from the proximal and distal aorta during the implantation, a blood pressure increase is obviated or mitigated. Thus, a unique advantage of the present prosthesis is that it can be used even in multiple places of the aorta when more parts of the aorta are showing thrombus formation.

### BRIEF DESCRIPTION OF THE DRAWINGS

Embodiments of the present invention will be described with reference to the accompanying drawings, in which:

FIG. 1 illustrates a summary of the various categories of aortic disease;

FIGS. 2a-2e illustrate various categories of dissection of the aorta;

FIG. 3 illustrates a perspective view of a preferred embodiment of the present endovascular prosthesis;

FIG. 4 illustrates a schematic, cross-sectional view of the human heart and various anatomy connected thereto;

FIGS. 5-13 illustrate various views of a preferred embodiment of the present endovascular prosthesis being deployed to occlude a Class 4 aortic dissection (in these Figures, FIG. 11 is a section along line XI-XI in FIG. 10).

### BEST MODE FOR CARRYING OUT THE INVENTION

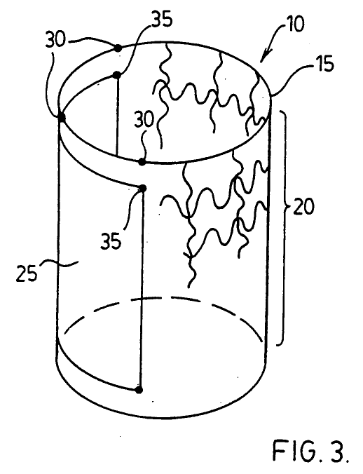

Thus, with reference to FIG. 3, there is illustrated an endovascular prosthesis 10. An endovascular prosthesis 10 comprises a tubular wall 15. Tubular wall 15 comprises a porous section shown generally at 20 and a non-porous section 25.

As will be appreciated by those of skill in the art, the terms “porous” and “non-porous” are used throughout the present specification in a relative sense. Thus, the term “non-porous” section is intended to mean a section of expandable prosthesis 10 which will cause thrombosis or clotting of bodily fluid (e.g., blood) located exteriorly adjacent to expandible prosthesis 10. Specifically, most aortic diseases subsist by receiving a regular flow of bodily fluid (e.g., blood). The aortic disease may be effectively occluded by impeding this regular fluid of bodily fluid. Thus, those of skill in the art will recognize that the “non-porous” section of expandable prosthesis 10 need not necessarily be fluid impermeable provided it provides sufficient impedance to the flow of bodily fluid therethrough.

Porous section 20 may be any conventional stent design which is preferably optimized to facilitate navigation of prosthesis 10 to the target site in the anatomy. The preferred design for non-porous section 20 is that disclosed in the Penn et al. International patent applications referred to above. Of course, those of skill in the art will recognize that the present endovascular prosthesis is not restricted to the use of the specific stent designs for porous section 20 and that any generally suitable stent design may be used.

As illustrated, non-porous section 25 is disposed on tubular wall 15. The nature of the material used in non-porous section 25 is not particularly restricted provided that it is generally biocompatible and that the physical nature thereof does not impede delivery, deployment and general efficacy of the endovascular prosthesis after it has been implanted.

In one embodiment, non-porous section 25 comprises a sheet material such as Dacron™, Gortex™, other polymeric materials, bovine pericardium and the like. The nature of the material used for this purpose is not particularly restricted. Non-porous section may also be derived from a silicone-based material such as those commercially available from NuSil Technology (Carpenteria, Calif.). A non-limiting example of such material is derived from a silicone-based dispersion commercially available from NuSil Technology under trade name MED-6640. This material is usually obtained as a liquid dispersion in an organic insolvent such as xylene. The dispersion may be used as such or the viscosity thereof may be altered as desired by addition of further solvent.

Preferably, the cover material is attached to an otherwise tubular stent structure. The means by which attachment may be achieved is not particularly restricted. For example, the cover material could be fixed to the appropriate spot on the stent using a suitable adhesive. Alternatively, the cover material could be sewn onto the stent. Those of skill in the art will conceive of a number of other means by which the cover material may be fixed to the stent structure.

In another embodiment, non-porous section 25 may be made of the same material as porous section 20 but preferably suitably modified to comprises a number of slits, microcuts, slots, apertures and the like to reconcile the feature of impeding bodily fluid (e.g., blood) therethrough with the feature of rendering non-porous section 25 sufficiently flexible so as to permit delivery and deployment of expandable prosthesis 10.

As shown in the embodiment illustrated in FIG. 3, a portion of porous section 20 is disposed both distal and proximal with respect to non-porous section 25. Those of skill in the art will recognize that it is possible to dispose non-porous section 25 on tubular wall 15 in a manner such that one or both of the proximal and distal edges of the cover material are aligned with the proximal and distal edges, respectively of tubular wall 15. Further, it is possible to vary the design of porous section 20 in the regions which surround the proximal distal edges of the cover material compared to the remainder of porous section 20 of tubular wall 15.

With further reference to FIG. 3, endovascular prosthesis further comprises a first set of radiopaque markers 30 which are disposed on the distal edge of tubular wall 15. Further, a second set of radiopaque markers 35 are disposed at points along the distal edge of non-porous section 25. The use of such radiopaque markers facilitates correct placement of endovascular prosthesis 10 as will be described in more detail hereinbelow. The nature of radiopaque markings 30,35 is not particularly restricted. For example, radiopaque markers 30,35 may be made from gold or any other material which is opaque to X-ray radiation.

Preferably, non-porous section 25 spans a radial arc of from about 90° to about 270°, more preferably from about 150° to about 250°, most preferably to about 180° to about 240°, of an annular portion of tubular wall 15.

Further, the longitudinal length of endovascular prosthesis 10 is selected to correspond to the length of anatomy in which the device will be deployed. For example, if the endovascular prosthesis is to be deployed in the descending aorta, it is appropriate for a non-porous section to have longitudinal length of in the range of from about 2 cm to about 30 cm, more preferably from about 2 cm to about 25 cm, most preferably from about 2 cm to about 20 cm. In this preferred embodiment, the overall length of the expandable prosthesis would be more than this since it is preferred to have porous sections on opposite sides of the distal and proximal edges of the non-porous section.

Preferably, tubular wall 15 is constructed from a plastically deformable material such as stainless steel, tantalum or the like. Alternatively, the plastically deformable material could be made from a radioopaque composite material such as that in described in U.S. Pat. No. 5,858,556 [Eckert et al.]—this could obviate the use of radioopaque markers 30,35 described above. Generally, such devices are expanded with a balloon catheter.

Alternatively, it is possible to produce tubular wall 15 from a so-called “shape memory alloy” which will expand when a certain temperature is reached. In this embodiment, the material may be a metal alloy (e.g., Nitinol) capable of self-expansion at a temperature of at least 30° C., preferably in the range of about 30° to about 40° C.

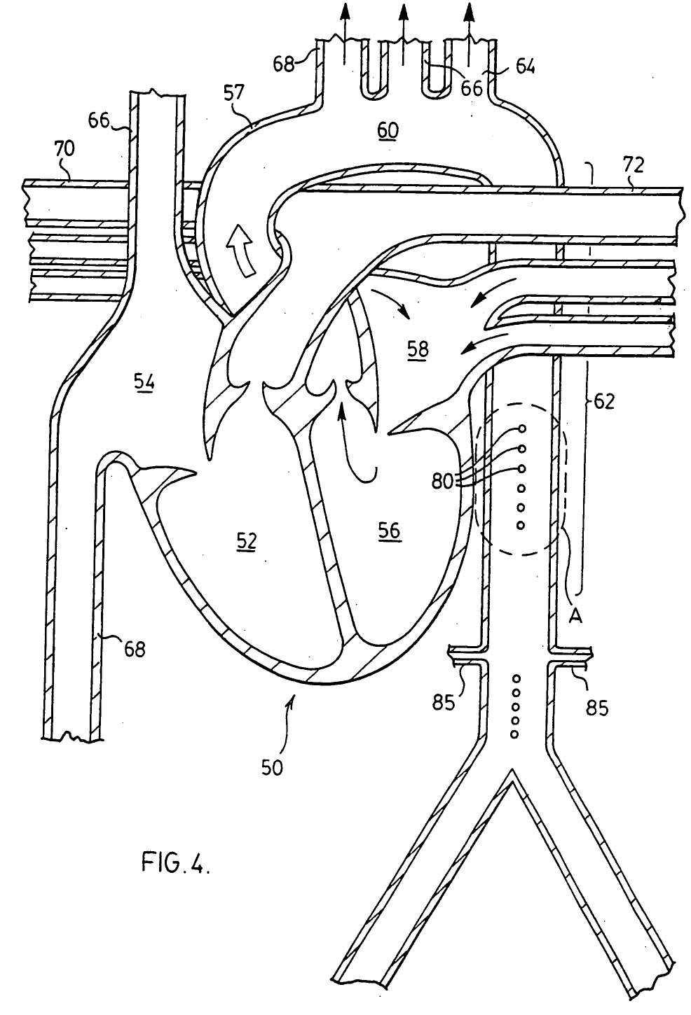

With reference to FIG. 4, it is appropriate to set out some basic anatomical terms which are used throughout the present specification. Thus, there is illustrated a heart 50. Heart 50 comprises right ventricle 52, right atrium 54, left ventricle 56 and left atrium 58.

Emanating from heart 15 is ascending aorta 57 which transitions into aortic arch 60 and then descending aorta 62. Emanating from aorta arch 60 is left subclavian artery 64, left common carotid artery 66 and innominate artery) 68.

As illustrated, superior vena cava 66 and inferior vena cava 68 are in communication with right atrium 54. Further, right innominate vein 70 and left innominate vein 72 are in communication with right ventricle 52.

As shown, descending aorta 62 comprises a plurality of side branches 80. It is important during use of the present endovascular prosthesis that these side branches not be occluded as this can result in paralysis of the patient.

As shown, renal arteries 85 are in communication with descending aorta 62 and also should not be occluded during catheterization techniques employing the present endovascular prosthesis.

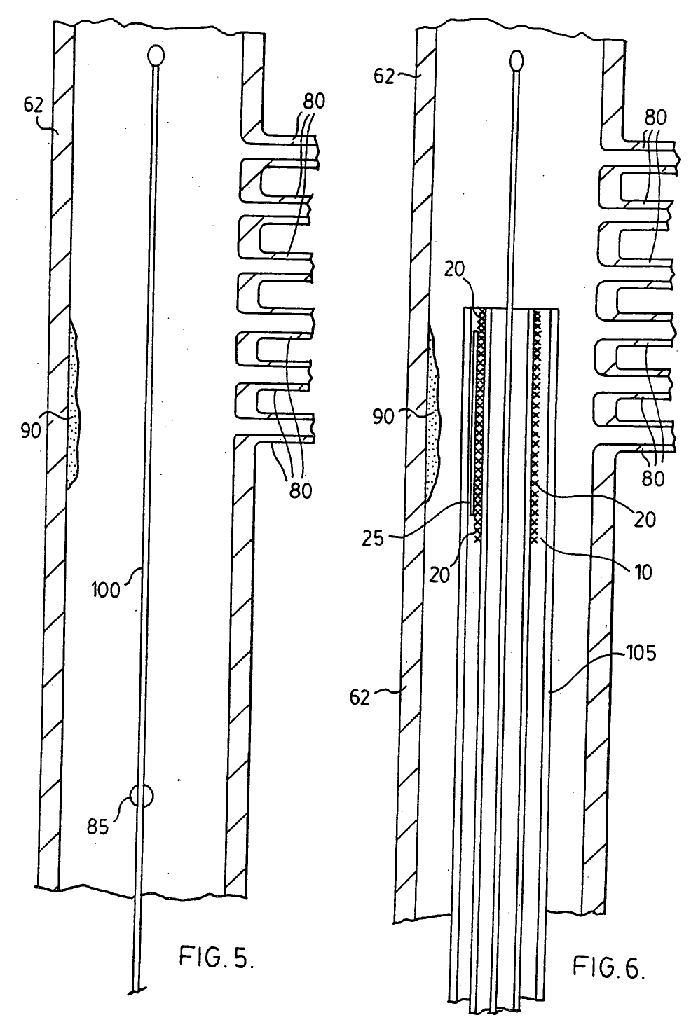

With reference to FIGS. 5-11, deployment of endovascular prosthesis 10 will be illustrated. In the illustrated example, tubular wall 15 of endovascular prosthesis 10 is constructed from a plastically deformable material such as Nitinol™.

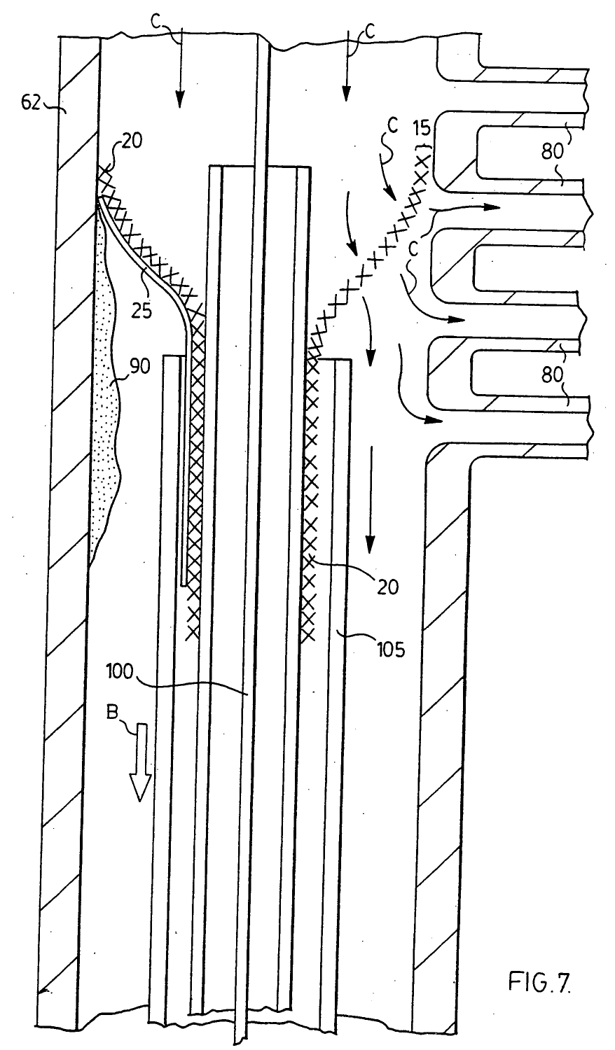

Thus, with reference to FIGS. 5 and 6, there is illustrated an enlarged portion of the descending aorta 62 illustrated in Region A in FIG. 4. In the illustrated embodiment, a blockage 90 has formed on a wall of descending aorta 62 opposite side branches 80. Blockage 90 may be manifested as a Class 4 dissection (e.g., aortic sclerosis).

As illustrated, the first step in deploying the endovascular prosthesis 10 involves a conventional catheterization step of navigating a guidewire 100 such that the distal end thereof is distal blockage 90. Next, it is conventional to insert a guide catheter to appoint just proximal blockage 90. For clarity, this step is not shown. Thereafter, a sheath 105 encompassing endovascular prosthesis 10 is navigated such that non-porous section 25 of endovascular prosthesis 10 is aligned with blockage 90 and porous section 20 of endovascular prosthesis 10 is aligned with side branches 80.

Once endovascular prosthesis 10 is correctly positioned, sheath 105 is retracted in a direction of arrow B as shown in FIG. 7. This results in exposure of endovascular prosthesis 10 to blood flow at the appropriate temperature which causes tubular wall 15 of endovascular prosthesis 10 to “self expand”.

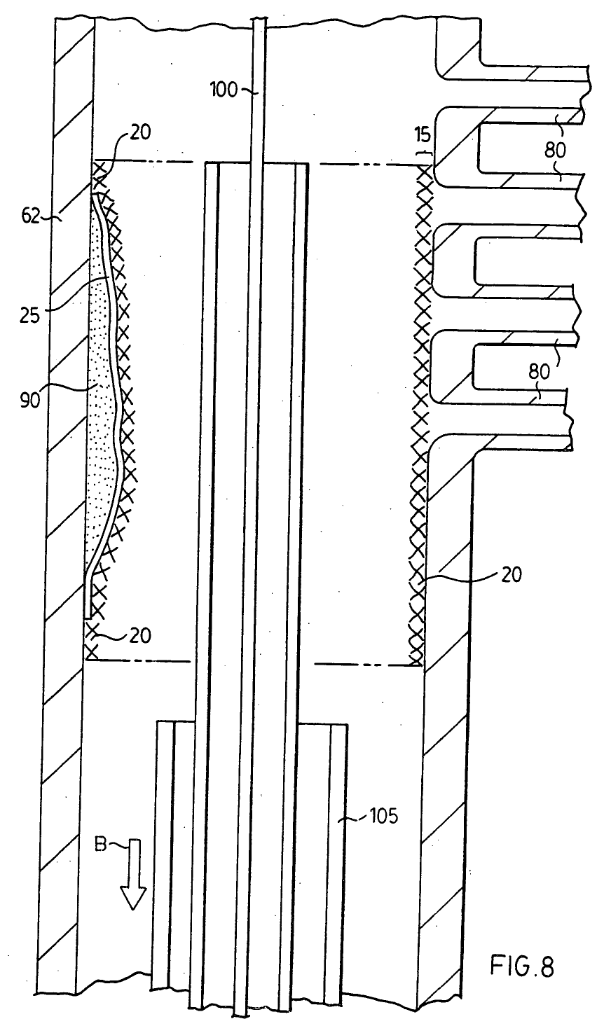

Thus, as shown in FIG. 8, when sheath 105 has been retracted to expose the entire endovascular prosthesis 10, the latter fully expands with non-porous section 25 occluding blockage 90 while permitting blood flow through porous section 20 and into side branches 80 (arrows C) see, also, FIG. 10.

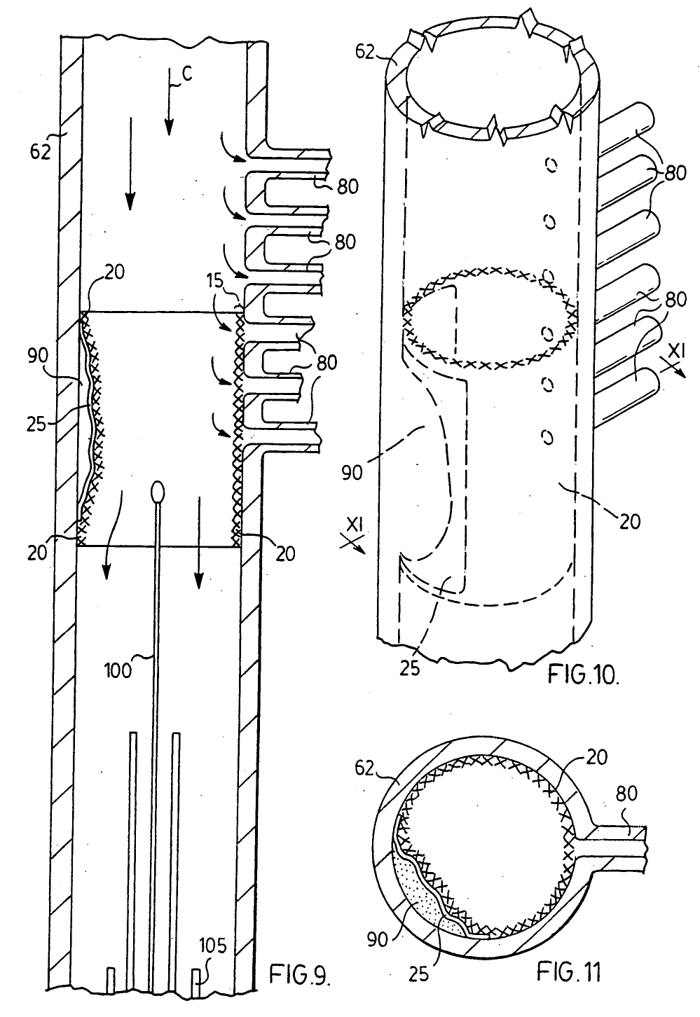

With reference to FIGS. 10 and 11, the deployed endovascular prosthesis 10 is shown in perspective and sectional views, respectively.

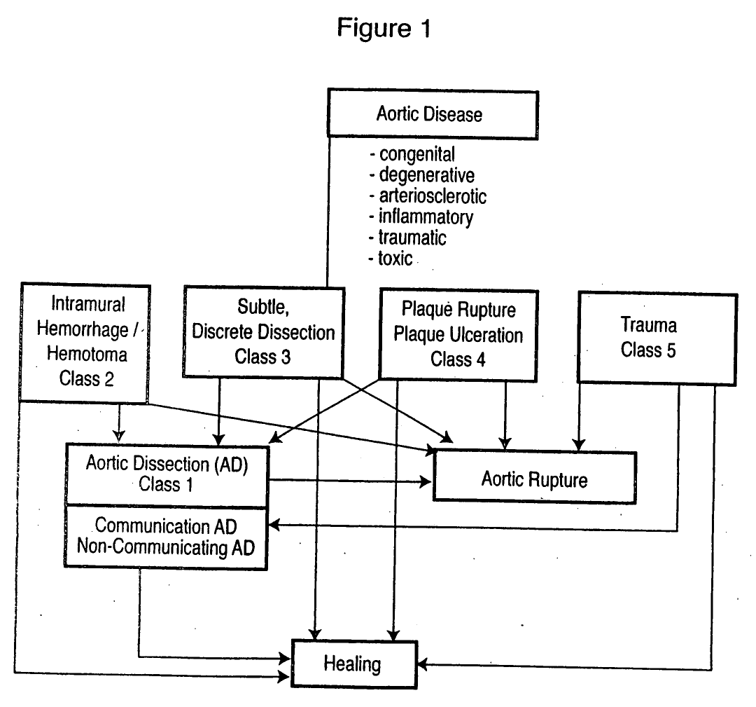

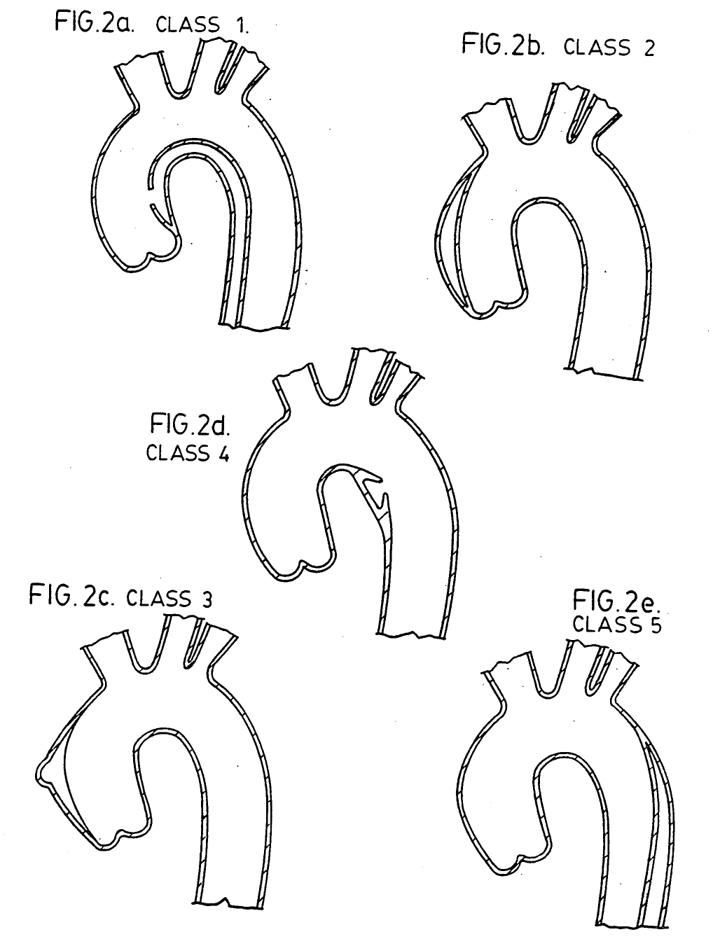

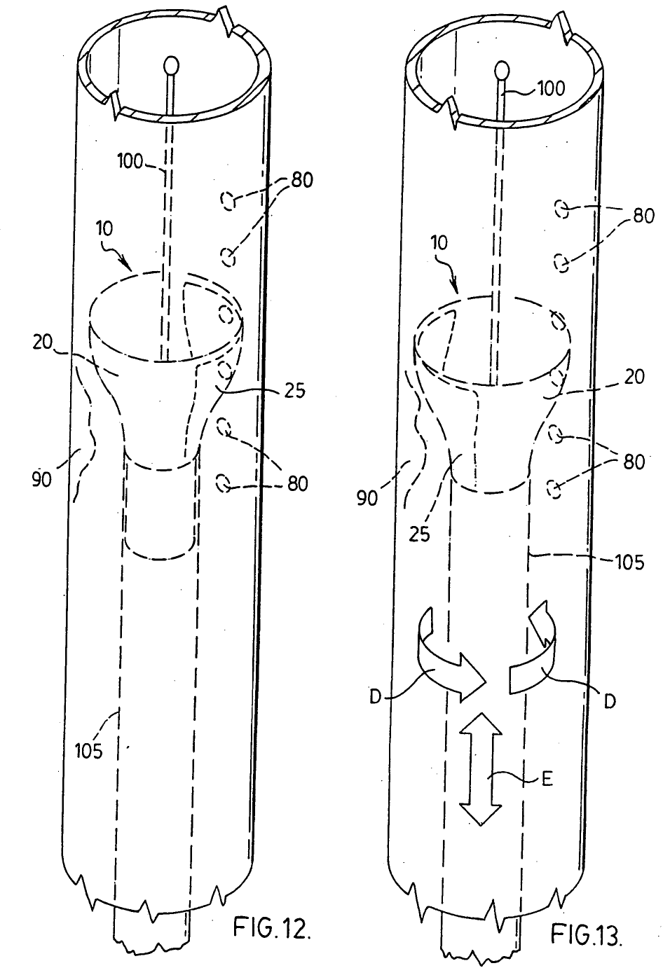

With reference to FIGS. 12 and 13, there is shown, in schematic form, adjustment of positioning of endovascular prosthesis 10 before it is fully deployed. Thus, as described with reference to FIG. 1, it is preferred that, if tubular wall 15 of endovascular prosthesis 10 is constructed from a radio transparent material, discreet portions thereof be marked with a radiopaque material.

In the embodiment illustrated in FIG. 12, if endovascular prosthesis 10 were fully deployed as illustrated, non-porous section 25 would occlude side branches 80 resulting in significant risk to the patient. In such a situation, it is possible to alter orientation of endovascular prosthesis 10 (and by inference, non-porous 25) by rotating sheath 105 in the direction of arrows D and or extending/retracting sheath 105 in the direction of arrow E—see FIG. 13. Once non-porous section 25 is properly positioned with respect to blockage 90, sheath 105 is retracted as described above to deploy endovascular prosthesis 10.

The present endovascular prosthesis may further comprise a coating material thereon. The coating material may be disposed continuously or discontinuously on the surface of the prosthesis. Further, the coating may be disposed on the interior and/or the exterior surface(s) of the prosthesis. The coating material can be one or more of a biologically inert material (e.g., to reduce the thrombogenicity of the prosthesis), a medicinal composition which leaches into the wall of the body passageway after implantation (e.g., to provide anticoagulant action, to deliver a pharmaceutical to the body passageway and the like) and the like.

The present endovascular prosthesis is preferably provided with a biocompatible coating in order to minimize adverse interaction with the walls of the body vessel and/or with the liquid, usually blood, flowing through the vessel. The coating is preferably a polymeric material, which is generally provided by applying to the prosthesis a solution or dispersion of preformed polymer in a solvent and removing the solvent. Non-polymeric coating material may alternatively be used. Suitable coating materials, for instance polymers, may be polytetraflouroethylene or silicone rubbers, or polyurethanes which are known to be biocompatible. Preferably, however, the polymer has zwitterionic pendant groups, generally ammonium phosphate ester groups, for instance phosphoryl choline groups or analogues thereof. Examples of suitable polymers are described in International Publication Numbers WO 93/16479 and WO 93/15775. Polymers described in those specifications are hemo-compatible as well as generally biocompatible and, in addition, are lubricious. It is important to ensure that the surfaces of the prosthesis are completely coated in order to minimize unfavourable interactions, for instance with blood, which might lead to thrombosis in the parent vessel and/or endoleaks therethrough.

This good coating can be achieved by suitable selection of coating conditions, such as coating solution viscosity, coating technique and/or solvent removal step.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. An endovascular prosthesis for implantation in a body passageway, the prosthesis comprising an elongate tubular wall, the tubular wall comprising an annular portion for occlusion of a section of the body passageway, the annular portion comprising a first porous section and a non-porous section.

2. The endovascular prosthesis defined in claim 1, wherein the tubular wall comprises a second porous section adjacent the annular portion.

3. The endovascular prosthesis defined in claim 1, wherein the tubular wall comprises a third porous section adjacent the annular portion.

4. The endovascular prosthesis defined in claim 1, wherein the tubular wall comprises a second porous section disposed adjacent one side of the annular portion and a third porous section adjacent an opposed side of the annular portion.

5. The endovascular prosthesis defined in claim 4, wherein the second porous section and the third portion section are interconnected by the first porous section.

6. The endovascular prosthesis defined in any one of claims 1-5, wherein the tubular wall is constructed from a plastically deformable material.

7. The endovascular prosthesis defined in claim 6, wherein the plastically deformable material comprises stainless steel.

8. The endovascular prosthesis defined in claim 6, wherein the plastically deformable material comprises a laminar structure.

9. The endovascular prosthesis defined in claim 8, wherein the laminar structure comprises a layer of plastically deformable material and a layer of radioopaque material.

10. The endovascular prosthesis defined in any one of claims 1-5, wherein the tubular wall is constructed from a self-expanding material.

11. The endovascular prosthesis defined in claim 10, wherein the self-expanding material comprises a shape memory alloy.

12. The endovascular prosthesis defined in any one of claims 1-11, wherein the non-porous section radially spans from about 90° to about 270° of the annular portion.

13. The endovascular prosthesis defined in any one of claims 1-11, wherein the non-porous section radially spans from about 150° to about 250° of the annular portion.

14. The endovascular prosthesis defined in any one of claims 1-11, wherein the non-porous section radially spans from about 180° to about 240° of the annular portion.

15. The endovascular prosthesis defined in any one of claims 1-14, wherein the non-porous section extends longitudinally a distance in the range of from about 2 cm about 30 cm.

16. The endovascular prosthesis defined in any one of claims 1-14, wherein the non-porous section extends longitudinally a distance in the range of from about 2 cm about 25 cm.

17. The endovascular prosthesis defined in any one of claims 1-14, wherein the non-porous section extends longitudinally a distance in the range of from about 2 cm about 20 cm.

18. The endovascular prosthesis defined in any one of claims 1-17, wherein the non-porous section comprises a cover material disposed over a fourth porous section.

19. The endovascular prosthesis defined in any one of claims 1-17, wherein the non-porous section comprises a cover material disposed connected to the first porous section.

20. The endovascular prosthesis defined in any one of claims 18-19, wherein the cover material comprises a layer of polymer material.

21. The endovascular prosthesis defined in any one of claims 1-17, wherein the non-porous section comprises a plurality of slits disposed in the tubular wall.

22. The endovascular prosthesis defined in any one of claims 1-17, wherein the non-porous section comprises a plurality of microcuts disposed in the tubular wall.

23. The endovascular prosthesis defined in any one of claims 1-24, wherein the tubular wall comprises at least one radioopaque marker.

24. The endovascular prosthesis defined in any one of claims 1-24, wherein the tubular wall comprises a pair of radioopaque markers disposed at opposed ends of the tubular wall.

25. The endovascular prosthesis defined in any one of claims 1-24, wherein the tubular wall comprises a pair of radioopaque markers disposed at opposed ends of the non-porous section.

26. A method for endovascular blocking of an aortic disease condition in a body passageway of a patient with an endovascular prosthesis comprising an elongate tubular wall, the tubular wall comprising an annular portion for occlusion of the aortic disease condition, the annular portion comprising a first porous section and a non-porous section, the method comprising the steps of:
disposing the prosthesis in a catheter;
inserting the prosthesis and catheter within a body passageway by catheterization of the body passageway;
translating the prosthesis and catheter to a target body passageway at which the aortic disease condition is located;
positioning non-porous section such that it is substantially aligned with the aortic disease condition;
exerting a radially outward expansive force on the tubular wall such that the tubular wall is urged against the target body passageway; and
urging the non-porous section against the aortic disease condition thereby blocking the aortic disease condition.

27. The method defined in claim 26, wherein aortic disease condition comprises aortic dissection.

28. The method defined in claim 26, wherein aortic disease condition comprises blunt chest trauma.

29. The method defined in claim 26, wherein aortic disease condition comprises aortic sclerosis.

30. The method defined in any one of claims 26-29, wherein the tubular wall comprises a second porous section adjacent the annular portion.

31. The method defined in any one of claims 26-29 wherein the tubular wall comprises a third porous section adjacent the annular portion.

32. The method defined in any one of claims 26-29, wherein the tubular wall comprises a second porous section disposed adjacent one side of the annular portion and a third porous section adjacent an opposed side of the annular portion.

33. The method defined in claim 32, wherein the second porous section and the third portion section are interconnected by the first porous section.

34. The method defined in any one of claims 26-33, wherein the tubular wall is constructed from a plastically deformable material.

35. The method defined in claim 34, wherein the plastically deformable material comprises stainless steel.

36. The method defined in claim 34, wherein the plastically deformable material comprises a laminar structure.

37. The method defined in claim 36, wherein the laminar structure comprises a layer of plastically deformable material and a layer of radioopaque material.

38. The method defined in any one of claims 26-33, wherein the tubular wall is constructed from a self-expanding material.

39. The method defined in claim 38, wherein the self-expanding material comprises a shape memory alloy.

40. The method defined in any one of claims 26-39, wherein the non-porous section radially spans from about 90° to about 270° of the annular portion.

41. The method defined in any one of claims 26-39, wherein the non-porous section radially spans from about 150° to about 250° of the annular portion.

42. The method defined in any one of claims 26-39, wherein the non-porous section radially spans from about 180° to about 240° of the annular portion.

43. The method defined in any one of claims 26-42, wherein the non-porous section extends longitudinally a distance in the range of from about 2 cm about 30 cm.

44. The method defined in any one of claims 26-42, wherein the non-porous section extends longitudinally a distance in the range of from about 2 cm about 25 cm.

45. The method defined in any one of claims 26-42, wherein the non-porous section extends longitudinally a distance in the range of from about 2 cm about 20 cm.

46. The method defined in any one of claims 26-45, wherein the non-porous section comprises a cover material disposed over a fourth porous section.

47. The method defined in any one of claims 26-45, wherein the non-porous section comprises a cover material disposed connected to the first porous section.

48. The method defined in any one of claims 46-47, wherein the cover material comprises a layer of polymer material.

49. The method defined in any one of claims 26-48, wherein the tubular wall comprises at least one radioopaque marker.

50. The method defined in any one of claims 26-48, wherein the tubular wall comprises a pair of radioopaque markers disposed at opposed ends of the tubular wall.

51. The method defined in any one of claims 26-48, wherein the tubular wall comprises a pair of radioopaque markers disposed at opposed ends of the non-porous section.
